(19) United States
(12) Patent Application Publication
(10) Pub. No.: US 2004/0156887 A1
(43) Pub. Date: Aug. 12, 2004

(54) Stabilized dispersion of phytosterol in oil

(76) Inventor: Nicolas Auriou, Bern (CH)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/477,802

(22) PCT Filed: Jun. 7, 2002
(86) PCT No.: PCT/EP02/06281

(30) Foreign Application Priority Data
Jun. 8, 2001 (GB) 0114014.4

(51) Int. Cl. A61K 9/127; A61K 31/56

(52) U.S. Cl. 424/450; 514/170

(57) ABSTRACT

The invention relates to a method for avoiding recrystallization of phytosterols from oil, involving use of a synergistic stabilizing combination of free fatty acids and phospholipids. A further benefit of these additives is that they allow dispersion of phytosterols in oil to be carried out at a temperature lower than the melting point of the phytosterols, thereby facilitating incorporation of phytosterols into foodstuffs and medicaments.

### FIELD OF THE INVENTION

The present invention relates to a method of dissolving or dispersing phytosterols in an oil matrix, to the products of that method, and to medical and nutritional uses of such products.

### BACKGROUND OF THE INVENTION

Phytosterols are widely known to deliver health benefits by oral administration. However, their physicochemical properties complicate incorporation of these molecules into standard food or pharmaceutical matrices. In particular, experience has shown that the only way to achieve a homogeneous dispersion of phytosterols in a lipid medium is through a melting step. Phytosterols are waxy substances with high melting points (usually in the range of about 130-150° C.), and at these elevated temperatures the phytosterol itself and other lipid components of the mixture are vulnerable to oxidation.

It has also proved to be problematic to solubilize large amounts of phytosterol in an oil or fat matrix, with the result that orally-administered products containing clinically effective levels of phytosterols can have an unpleasant gritty or waxy mouth-feel. Furthermore, after short periods of storage, in particular when subjected to low temperatures (0 to 25° C.), and in the presence of moisture, phytosterol-supplemented oils develop a cloudy appearance due to precipitation of phytosterol crystals. The cloudiness of the oil decreases the visual appeal of the product, and the health benefits are reduced once the phytosterol has come out of solution because the product is no longer homogenous.

In the past, emulsifiers have been employed in an attempt to dissolve phytosterols in oils, and to stabilize the resulting dispersion against crystallization. For instance, free fatty acids have been described as suitable agents for counteracting this stability problem. However, the concentration of free fatty acids in edible products is controlled in many countries due to health concerns (for example, the European Commission prescribes a maximum concentration of 3%l), and at such concentrations the solubility problem is not completely resolved. Furthermore, at higher fatty acid concentrations the flavour of edible oils is adversely affected.

Other emulsifiers proposed for use as stabilizers are not capable of preventing crystallization under demanding storage conditions, such as exposure to humid air and refrigeration.

It is an object of the current invention to present a new, effective and economical solution to the phytosterol stability problem in an oil matrix, and thereby to enable provision of edible oils containing phytosterols in convenient formats and packaging with long-term shelf-stability.

### SUMMARY OF THE INVENTION

In a first aspect of the invention there is provided a process for dispersing phytosterols in oil, comprising mixing together said oil, said phytosterols, free fatty acids, and phospholipid.

In a second aspect of the invention there is a provided a method for stabilizing a dispersion of phytosterols in oil, comprising adding free fatty acids and phospholipid to the dispersion.

In a third aspect of the invention there is provided a dispersion of phytosterol in oil comprising at least 3% by weight phytosterols, at least 0.3% by weight free fatty acids and at least 0.15% by weight phosphatidylcholine, based on the total weight of the dispersion.

In a further aspect of the invention there is provided a dispersion of phytosterol in oil which comprises at least 3% by weight phytosterols, at least 0.3% by weight free fatty acids and at least 0.6% by weight lecithin, based on the total weight of the dispersion.

In another aspect of the invention there is provided a medicament, nutritional formulation or cosmetic composition comprising a dispersion of phytosterols in oil comprising at least 3% by weight phytosterols, at least 0.3% by weight free fatty acids, and at least 0.15% by weight phosphatidylcholine, based on the total weight of the dispersion.

In a further aspect of the invention there is provided a medicament, nutritional formulation or cosmetic composition comprising a dispersion of phytosterols in oil comprising at least 3% by weight phytosterols, at least 0.3% by weight free fatty acids, and at least 0.6% by weight lecithin, based on the total weight of the dispersion.

In a yet further aspect of the invention there is provided a use as a medicament of a dispersion of phytosterols in oil comprising at least 3% by weight phytosterols, at least 0.3% by weight free fatty acids, and at least 0.15% by weight phosphatidylcholine, based on the total weight of the dispersion.

In a yet further aspect of the invention there is provided a use as a medicament of a dispersion of phytosterols in oil comprising at least 3% by weight phytosterols, at least 0.3% by weight free fatty acids, and at least 0.6% by weight lecithin, based on the total weight of the dispersion.

In yet another aspect of the invention there is provided a use of a dispersion of phytosterols in oil comprising at least 3% by weight phytosterols, at least 0.3% by weight free fatty acids, and at least 0.15% by weight phosphatidylcholine, based on the total weight of the dispersion, in the manufacture of a medicament or nutritional formulation for the treatment or prevention of any of: hypercholesterolemia, hypertriglyceridemia, coronary heart disease, diabetes, atherosclerosis, inflammation, osteoarthritis, Alzheimer's disease, breast cancer, colon cancer, and benign prostatic hyperplasia.

In a further aspect of the invention there is provided a use of a dispersion of phytosterols in oil comprising at least 3% by weight phytosterols, at least 0.3% by weight free fatty acids, and at least 0.6% by weight lecithin, based on the total weight of the dispersion, in the manufacture of a medicament or nutritional formulation for the treatment or prevention of any of: hypercholesterolemia, hypertriglyceridemia, coronary heart disease, diabetes, atherosclerosis, inflammation, osteoarthritis, Alzheimer's disease, breast cancer, colon cancer, and benign prostatic hyperplasia

### DETAILED DESCRIPTION OF THE INVENTION

A striking stabilizing effect on dispersions of phytosterols in edible oil has been achieved through a synergistic combination of added free fatty acids and lecithin. We have observed that the level of phospholipids, e.g. acetone insolubles,—and in particular the level of phosphatidylcholine (PC)—in lecithin is a determining factor in the efficacy of lecithin as a stabilizing agent for phytosterols in oil or fat.

In general, the more hydrophilic the lecithin preparation the better the stabilizing effect, and this correlates positively with the PC, and/or lyosphosphatidylcholine (LPC), content of the lecithin. Although this makes it desirable to use a lecithin preparation rich in PC/LPC for stabilization of phytosterols in oil, there is a direct correspondence between PC/LPC content and price, and therefore it can be expensive to obtain sufficient phytosterol stabilisation when relying on lecithin alone for that effect. However, by exploiting the synergy between PC and free fatty acids we have discovered that it is possible to obtain surprisingly effective stabilization at relatively low concentrations of these additives, and therefore inexpensively. Table 1 shows that the stabilizing effect of the combination of free fatty acids and lecithin together can be several fold higher than that which might be expected on an additive basis.

By dispersing or dissolving phytosterols in oil using the method of the present invention it has been possible not only to maximize the concentration of phytosterols that can be dispersed and maintained in solution in oil over time, but also to achieve dispersion economically and without the need for a step involving melting of the phytosterol. The resulting products have acceptable organoleptic properties and are suitable for ingestion in the form of diverse oil-based nutritional and pharmaceutical formulations.

We have observed that the difficulties experienced in dissolving phytosterols in oils are primarily a function of the water content of the oil, and not due to poor solubility of phytosterols in oil per se at temperatures below their melting points. As a consequence of this realization we decided to test the incorporation of high concentrations of phytosterol into oil at temperatures far below the melting point of these phytosterols using the synergistic stabilizers of the invention, and found this method to be technically feasible.

The invention allows fats containing phytosterols to be stored and sold in an extended range of packaging formats and materials. It is not essential that the container is airtight, so a variety of packaging materials with different closing and sealing means can be used. The useful life of the product once any airtight seal has been broken is also extended by means of the inventive method disclosed herein, thereby making the product more attractive and cost-effective for the consumer. Another benefit of the improvements in product stability is that the phytosterol-containing oils can be stored at low temperatures. Thus, for example it is possible to refrigerate foodstuffs containing these oils without risking significant precipitation of phytosterol crystals.

In general, the fats suited for use in the present invention include both animal and vegetable oils which are liquid at room temperature and solid fats which can be melted at moderate temperatures, for example at temperatures below 130° C. The terms “fat” and “oil” are used interchangeably herein to refer to this generic group of fats and oils. For preparation of foodstuffs, the fats employed as the matrix for dissolving phytosterols are any of those suited for human consumption, and particularly those which are liquid at room temperature and commonly used as salad, cooking or frying oils.

According to the invention, dispersion of phytosterol in oil can be carried out at any stage during processing or refining of the oil. For the purposes of describing the invention the terms dispersion, emulsion, suspension and dissolution are used interchangeably to describe creation of homogenous mixtures of phytosterols in oil. Such a mixture may also be defined herein as a phytosterol-enriched oil.

It is envisaged that the process of refining oil, e.g. the degumming step, could be adjusted in order to maintain the naturally high levels of free fatty acids and lecithin in the crude oil, e.g. roughly 4-8% by weight and 2-3% by weight, respectively, based on the total weight of the oil, in certain crude vegetable oils, thereby eliminating the need to add supplemental stabilizing agents to the refined oil. However, in general the stabilizing agents are added to the refined oil in the latter stages of processing before the oil is ready to be consumed. It is also foreseen that addition of phytosterol to oil could be delayed until immediately prior to use of the oil in food preparation, when mild heating could be applied to promote dissolution. In that case the oil could be provided already supplemented with free fatty acids and PC or lecithin, or alternatively the phytosterol and stabilizers could be added together to the oil prior to use.

Oils, e.g. triglyceride oils, suited for use in the invention include sunflower, corn, rapeseed, peanut, grapeseed, olive, cotton seed, linseed, sesame seed, wheat germ, palm kernel, soybean, avocado, canola, fish oils and other oils conventionally used in the food and pharmaceutical industries. Dairy fat, shortenings, and hydrogenated, fractionated and interesterified oils may also be employed. In preparing the dispersion of phytosterols in oil, the proportion of oil is generally 50-97% by weight, preferably 75-95% by weight, and most preferably 80-90% by weight, based on the total weight of the dispersion.

In the present context the generic term “sterol” or “phytosterol” is intended to encompass any member of the family of free phytosterols, e.g. non-hydrogenated, and phytostanols, e.g. saturated or hydrogenated phytosterols, esters and glycosides or other derivatives thereof, including isomers, and any mixture or combination thereof.

The phytosterols used in the invention may be chemically synthesized, or may be derived from natural sources, including plants sources such as avocado, soy, rice bran, tall oil pitch or soap, shea nut, coconut, and plant oils, for example rapeseed, soya, maize, sunflower and sesame oils. Some germ oils are very rich in phytosterols, wheat germ and oats being good examples. Non-exhaustive examples of plant sterols include sitosterol, stigmasterol, campesterol, brassicasterol, desmosterol, chalinosterol, poriferasterol, avenasterol, and clionasterol, and their corresponding esters and stanols.

The particle size of the phytosterols is not crucial to operation of the invention. However, dispersion in oil may be facilitated by using finely divided phytosterols, preferably where 95% of the particles have a size (diameter) of less than 100 μm, preferably less than 30 μm and most preferably less than 15 μm. The particle size distribution will usually be such that 90% of the particles are in the range 100 nm to 35 μm, more preferably 0.2 to 20 μm, and most preferably 0.5 to 15 μm. Particle mixtures with this size range can be prepared by standard milling or pulverization techniques, such as by use of an air mill, high energy hammermill, disc mill, or air filtration mill. Optionally, the phytosterol is milled in the presence of sugar, and the powdered phytosterol/sugar mix is used in the preparation of sweetened food or nutritional products.

A large amount of phytosterol can be permanently dispersed or dissolved in oil using the method of the invention, such that a small dose of the oil can confer a significant medical benefit on the consumer. For the purposes of influencing blood cholesterol levels it is usual to aim for a concentration of phytosterol in the finished product of between about 0.1% and 20% by weight, more preferably 1% to 15% by weight, and most preferably 4% to 10% by weight, for example 5 to 8% by weight, based on the total weight of the product. Therefore, if the finished product is the phytosterol-enriched oil in pure form, e.g. approximately 100% by weight oil, the aforementioned ranges also apply to the phytosterol content of the oil employed for dissolving the phytosterols. If the phytosterol-enriched oil contributes less than about 100% by weight of the product, for example if the phytosterol-enriched oil is used to prepare a yellow fat spread, a higher concentration of phytosterol may be dispersed in the oil component, e.g. up to about 50% by weight, or in the range 3 to 30% by weight, more preferably 5 to 25% by weight, even more preferably 8 to 15% by weight, and most preferably 6 to 12% by weight, based on the total weight of the oil.

The free fatty acids used as stabilizers can be any of those known in the art, and especially those disclosed in U.S. Pat. No. 3,865,939, which is incorporated herein by reference. In particular, saturated and unsaturated fatty acids having from 6 to 18 carbon atoms are preferred, some examples being oleic, linoleic, linolenic, stearic, palmitic, palmitoleic, hexanoic, lauric acids, and mixture thereof. Because of health and organoleptic concerns, for oral ingestion the concentration of free fatty acids in the dispersion of the invention, e.g phytosterol-enriched oil, will not normally exceed 3% by weight, based on the total weight of the dispersion. However, in terms of efficacy the concentration of free fatty acids in the dispersion will ideally lie in the range 0.3-15% by weight, more usually 0.5-5% by weight, and generally 1-3% by weight, based on the total weight of the dispersion.

Natural or chemically synthesized pure phospholipids, or derivatives thereof such as lysophospholipids, or any natural edible source of phospholipids may be selected for use in the synergistic stabilizing composition of the invention. PC is the preferred phospholipid; others include phosphatidyl serine (PS), phosphatidyl ethanolamine (PE), phosphatidyl inositol (PI), N-acylphosphatidyl ethanolamine (NAPE), phosphatidyl glycerol, phosphatidic acid and lysophosphatides. Because of its widespread use in foods, lecithin, e.g. lecithin from egg yolk or soya, or E322, is a convenient source of PC and other phospholipids. The phospholipid content of lecithin can vary widely. Preferred lecithins, e.g. lecithin preparations, are those having a high PC content, at least 30% by weight PC, optionally at least 40% by weight PC, especially at least 60% by weight, or at least 90% by weight, or more preferably lecithins, e.g. lecithin preparations, having a PC content within the range 30-98% by weight, based on the total weight of the lecithin. Since hydrophilic lecithin preparations are superior stabilizing agents these are preferred for use in performing the process of the invention. The Hydrophile-Lipophile Balance (HLB) of the lecithin will ideally be in the range 4-10, preferably 6-8.

The PC content of the dispersion made by the method of the invention will generally lie in the range 0.1-3% by weight, preferably 0.2-1% by weight, and most preferably 0.3-0.75% by weight, based on the total weight of the dispersion. Where lecithin is used, e.g. as the source of the PC, the concentration of lecithin in the dispersion commonly falls within the range 0.3-15% by weight, especially 0.6-10% by weight, preferably 0.5-5% by weight, most preferably 0.8-3% by weight. The larger the amount of phytosterol which must be dissolved in the oil, the greater the concentration of PC required to achieve dissolution, and therefore the higher the concentration of pure PC or lecithin which must be added at a fixed concentration of free fatty acids. The person skilled in the art can readily determine by simple experimentation the optimum amount of PC or other phospholipid required to dissolve the phytosterol at any particular concentration of phytosterol and free fatty acids.

The ratio (w/w) of free fatty acids to PC in the dispersion (in terms of weight percentages) will usually range from 15:1 to 1:10, preferably 10:1 to 1:1, and most preferably 5:1 to 2:1.

The step of dissolving phytosterols in oil is ideally carried out at a temperature below the melting temperature of the phytosterol, and generally below about 130° C. However, this is not obligatory, and if peroxidation is not a concern the product resulting from heating to temperatures of 130° C.-160° C. under vacuum or modified atmosphere, especially about 150° C., may have superior long-term resistance to phytosterol crystallization. Suitable heating temperatures below the melting temperature may lie in the range 30-130° C., or more usually 50-120° C., especially 90-110° C.

The dispersion step can be performed either without agitation, or by manual mixing, stirring, homogenization, high shear mixing, vortexing, sonicating or other means of agitation. Depending on the temperature of the mixture, dispersion may be completed in a matter of minutes, e.g. 5-15 minutes, or may be allowed to proceed over a number of hours, e.g. 0.5-10 hours, more often 1-3 hours.

Once a clear dispersion is achieved the dispersion of the invention, e.g the phytosterol-enriched oil, is allowed to cool down to room temperature or slightly above, for example, 15-35° C., preferably 20-30° C. Cooling may be accelerated by agitation of the dispersion, or by subjecting it to temperatures below 15-20° C.

No further treatment of the dispersion is required at this point, and the dispersion of the invention, e.g. the phytosterol-enriched oil may be packaged immediately. The dispersion of the invention, e.g. the phytosterol-enriched oil, may also be used to substitute for conventional edible oils in the preparation of nutritional products and medicaments. Typically, the dispersion of the invention, e.g phytosterol-enriched oil will constitute about 1 to 100% by weight of a finished nutritional or pharmaceutical product, especially about 5 to about 20% by weight.

Conventional food additives may be added directly to the phytosterol-enriched oil, or may be incorporated into food products made using the phytosterol-enriched oil. Examples of such additives include further stabilizing agents, e.g. emulsifiers, antioxidants, thickeners, salts, preservatives, flavouring agents, aromas, acidulants, food colours etc.

The phytosterol-enriched oils prepared according to the method of the invention can be used as cooking or salad oils. Alternatively they can be subjected to further processing steps, or incorporated into foodstuffs in the same manner as conventional oils. Emulsions of the type W/O or O/W can be made using the oils. The phytosterol-enriched oil is optionally hydrogenated for use in preparing yellow fat spreads such as margarines. Examples of foodstuffs which can be prepared using the dispersions of the invention, e.g. with phytosterol-enriched oils, include salad dressing, mayonnaise, shortenings, emulsified fat spreads and margarine, peanut butter, dips, breads, cookies, pies, cakes, crackers, noodles, pasta, sauces, soups, other savoury food products, including meat-based, pudding-type desserts, custard, chocolate, coffee whitener, dairy products such as cheese, ice cream, milk shakes, smoothies, yoghurt and yoghurt drinks, and formula diets. Since the invention permits very high concentrations of phytosterols to be homogeneously and stably dispersed in fats and oils it is technically feasible to make low fat varieties of all these products, which nevertheless deliver tangible health benefits. By “low fat product” is meant a product in which less than 5%, preferably less than 2%, of the total calories are contributed by lipids.

It is also possible to provide the oil in pharmaceutical form, e.g. encapsulated pharmaceutical form, or as a dietary supplement, for instance within a gelatin coating.

Further, it may be desirable to include the dispersion of the invention, e.g. phytosterol-enriched oil, in a cosmetic or topical pharmaceutical product, such as salves, creams, foams, lotions, gels, soaps, shampoos, and the like.

The pharmaceutical, food or drink products incorporating the dispersion of the invention, e.g. phytosterol-enriched oil, may be supplemented with other health-promoting ingredients, particularly ingredients known to have benefits for the cardiovascular system. Non-limiting examples are PUFAs, polyphenols, lipid-soluble antioxidants, e.g. tocopherol, tocotrienols, lycopene, as well as amino acids, dietary fibers, vitamins, minerals water-soluble antioxidants, e.g. ascorbate, and the like.

Pharmaceutical, food or beverage products incorporating the dispersion of the invention, e.g. phytosterol-enriched oil, can be consumed safely by anyone, but conveniently form part of the diet of those with a propensity for high blood cholesterol levels. According to one aspect of the invention a method is provided for preventing or treating high blood cholesterol levels comprising administering, to a person in need of such treatment, a dispersion of phytosterol in oil comprising at least 3% by weight phytosterols, at least 0.3% by weight free fatty acids and at least 0.15% by weight phosphatidylcholine or at least 0.6% by weight lecithin, based on the total weight of the dispersion. By “high blood cholesterol” is meant: over 200 mg/dl, especially over 240 mg cholesterol/dl blood; and/or a ratio total cholesterol/HDL of 5:1 or greater; and/or an LDL blood concentration of greater than 130 mg/dl, especially over 160 mg/dl. The pharmaceutical and nutritional products of the invention play a role in reducing blood cholesterol levels and thereby preventing cardiovascular disease and heart disease.

Aside from hypercholesterolemia, other indications for which dispersions of the invention, e.g phytosterol-enriched oil, may deliver a health benefit include: hypertriglyceridemia, coronary heart disease, diabetes, atherosclerosis, inflammation, osteoarthritis, Alzheimer's disease, breast cancer, colon cancer, and benign prostatic hyperplasia.

The amount and dosage regimen of the pharmaceutical, food or beverage products incorporating the dispersion of the invention is determined in the light of various relevant factors including the purpose of administration, the age, sex and body weight of individual subject and the severity of the subject's symptoms. Suitable daily doses of the dispersion according to the invention are in the range 0.1 to 100 g, preferably 1 to 75 g, more preferably 10 to 50 g and even more preferably 20 to 40 g.

The daily dosage ray correspond to a single unit dosage, or may be provided through multiple unit dosages. The exact amounts of the dispersion according to the invention may vary between wide limits and may be readily determined in conventional manner.

A further application of the invention is to provide topical cosmetic and pharmaceutical formulations incorporating the dispersions of the invention, e.g. phytosterol-enriched oils; these can include salves, creams, foams, lotions, gels, soaps, shampoos, and the like.

### EXAMPLE

### Comparison of the Stabilizing Effects of Free Fatty Acids, Lecithins, and Combinations Thereof

Table 1 depicts the results of an experiment comparing the stability of preparations of peanut oil containing 3% by weight added phytosterols.

The oil and phytosterol are mixed with lecithin and/or free fatty acids before heating up to either 100° C. until dissolution is complete, or heating up to 150° C. and then holding at that temperature for 5 minutes. The product is then cooled to 30° C. and dispensed into 250 ml glass bottles.

The open bottles are stored at 25° C. and 60% Relative Humidity and a daily record is kept to document when 2 or 3 crystals of minimum diameter 2 mm first appear.

TABLE I

| | | Heating | Period of |
|---|---|---|---|
| | Heating | Time | Stability |
| Trial Composition | Temperature | (min) | (days) |
| 3% Free Fatty Acids (FFA⁽¹⁾) | 150° C. | 5 | 3 |
| 1% E 145⁽²⁾ | 150° C. | 5 | 1 |
| 3% FFA + 1% E 145 | 150° C. | 5 | 60 |
| 1% E 135⁽³⁾ | 150° C. | 5 | 1 |
| 1% E 145 | 100° C. | 30 | 1 |
| 3% FFA + 1% E 135 | 100° C. | 10⁽⁴⁾ | 20 |
| 3% FFA + 1% E 145 | 100° C. | 30 | 25 |

⁽¹⁾FFA = Free Fatty Acids, being 66% by weight oleic acid (Merck)
⁽²⁾E145 is Epikuron 145V (Lucas Meyer), a deoiled lecithin emulsifier (97%) containing 45% by weight PC.
⁽³⁾E135 is Epikuron 135F (Lucas Meyer), a partially deoiled lecithin emulsifier (50%) containing 35% by weight PC.
⁽⁴⁾The shorter heating time required for dissolution is a reflection of the less hydrophilic nature of E135 in comparison with E145.

1. A process for dispersing phytosterols in oil, comprising mixing together said oil, said phytosterols, free fatty acids, and phospholipid.

2. A process according to claim 1 wherein the mixture is heated.

3. A process according to claim 2 wherein the mixture is heated to a temperature below the melting temperature of the phytosterols.

4. A process according to any preceding claim wherein the phospholipid comprises phosphatidylcholine.

5. A process according to any preceding claim wherein the phospholipid is provided in the form of lecithin.

6. A product obtainable by the process of any of claims 1 to 5.

7. A method for stabilizing a dispersion of phytosterols in oil, comprising adding free fatty acids and phospholipid to the dispersion.

8. A dispersion of phytosterol in oil comprising at least 3% by weight phytosterols, at least 0.3% by weight free fatty acids and at least 0.15% by weight phosphatidylcholine, based on the total weight of the dispersion.

9. A dispersion according to claim 8 wherein said phosphatidylcholine constitutes at least 0.25% by weight, based on the total weight of the dispersion.

10. A dispersion of phytosterol in oil comprising at least 3% by weight phytosterols, at least 0.3% by weight free fatty acids and at least 0.6% by weight lecithin, based on the total weight of the dispersion.

11. A dispersion according to claim 10 comprising at least 0.8% by weight lecithin, based on the total weight of the dispersion.

12. A dispersion according to any of claims 8 to 11 comprising at least 1% by weight free fatty acids, based on the total weight of the dispersion.

13. A medicament, nutritional formulation or cosmetic composition comprising the dispersion of any of claims 8 to 12.

14. A nutritional formulation according to claim 13 which is selected from: salad dressing, mayonnaise, shortenings, emulsified fat spreads and margarine, peanut butter, dips, breads, cookies, pies, cakes, crackers, noodles, pasta, sauces, soups, other savoury food products, e.g. vegetable- and meat-based, pudding-type desserts, custard, chocolate, coffee whitener, dairy products such as cheese, butter, ice cream, milk shakes, smoothies, yoghurt and yoghurt drinks, and formula diets.

15. Use of the dispersion of any of claims 8 to 12 as a medicament.

16. Use of the dispersion of any of claims 8 to 12 in the manufacture of a medicament or nutritional formulation for the treatment or prevention of any of: hypercholesterolemia, hypertriglyceridemia, coronary heart disease, diabetes, atherosclerosis, inflammation, osteoarthritis, Alzheimer's disease, breast cancer, colon cancer, and benign prostatic hyperplasia.
